(19) United States
(12) Patent Application Publication
(10) Pub. No.: US 2024/0286979 A1
(43) Pub. Date: Aug. 29, 2024

(54) VAPOR RECOVERY SYSTEM WITH COUNTERCURRENT AND CO-CURRENT ABSORBANT FLOW

(71) Applicant: Crown Iron Works Company, Blaine, MN (US)

(72) Inventors: Benjamin Wayne Floan, Andover, MN (US); Jeff Kraker, Blaine, MN (US); Jeff Garritsen, Forest Lake, MN (US); Wade Steven Martinson, Minneapolis, MN (US); Revathi Panuganti, Minneapolis, MN (US)

(21) Appl. No.: 18/589,694

(22) Filed: Feb. 28, 2024

Related U.S. Application Data
(60) Provisional application No. 63/487,467, filed on Feb. 28, 2023.

(51) Int. Cl. C07C 7/11; B01D 53/14; B01D 53/18

(52) U.S. Cl. CPC C07C 7/11; B01D 53/1406; B01D 53/1487; B01D 53/18; B01D 2252/20; B01D 2257/7022; B01D 2258/06

(57) ABSTRACT

A method of absorbing a solvent from a gas stream may involve flowing a gas stream comprising air and a hydrocarbon through an absorption column in a countercurrent direction with a first lean absorbent liquid to absorb solvent out of the gas stream and generate a first conditioned gas stream and a first rich absorbent liquid. The method can then involve flowing the first conditioned gas stream through the absorption column in a co-current direction with a second lean absorbent liquid to absorb solvent out of the first conditioned gas stream and generate a second conditioned gas stream having a reduced concentration of the hydrocarbon than the first conditioned gas stream and a second rich absorbent liquid.

### RELATED APPLICATION

This application claims priority to U.S. Provisional Patent Application No. 63/487,467, filed Feb. 28, 2023, the entire contents of which are incorporated herein by reference.

### TECHNICAL FIELD

This disclosure relates to gas absorption systems and, more particularly, to gas absorption systems for removing solvent vapor from non-condensable vent gases in liquid extraction processes.

### BACKGROUND

A variety of different industries use extractors to extract and recover substances entrained within solids. For example, producers of products from renewable organic sources use extractors to extract carbohydrates and/or oil from solid matter, such as soybeans, rapeseed, sunflower seed, peanuts, cottonseed, palm kernels, and corn germ. The matter is contacted with a solvent within the extractor, causing the desired product to be extracted from a surrounding cellular structure into the solvent. This can form a miscella stream containing solvent carrying material extracted from the cellular structure of the solid into the solvent as well as a residual solvent-wet solid material having undergone extraction.

After the matter is processed through the extractor, the miscella stream and the residual solvent-wet solid material can each be further processed to remove residual solvent from the two streams. The miscella stream can be vaporized, for example in a flash vessel and/or distillation column, to separate extracted material (e.g., oil) from the solvent. The solvent-wet solid material can also be desolventized to separate solvent and the residual extracted solid material. For example, the solvent-wet solid material can be processed in a desolventizer that vaporizes solvent from the extracted solid material.

Vent gases that are not condensable, such as air, may enter into the extraction system with the solids and mix with other gas streams. For example, gases streams produced through vaporization of the miscella stream and/or desolventization of the solvent-wet solid material may carry vaporized solvent. Each gas stream can be passed through a condenser to cool and condense the vaporized solvent for recovery and reuse. However, gas passing through the condenser may still carry a residual amount of solvent that needs to be removed before the vent gas can be discharged to the atmosphere. This non-condensable vent gas may be processed in an absorption column to absorb one or more non-condensable constituent components from the gas before discharging the gas to atmosphere. Increasing the efficiency of the absorption system can reduce the operating cost of the system and/or increase the amount of solvent removed from the vent gas before atmospheric discharge.

### SUMMARY

In general, this disclosure is directed to systems and techniques for absorbing a solvent from a gas stream, such as absorbing an organic solvent from a gas stream produced by one or more unit operations in a solvent extraction process. In some examples, an extraction system includes an extractor that contacts an oleaginous feedstock with an organic solvent to produce a miscella stream composed of solvent containing extracted oil and a residual solids stream composed of solvent-wet solids having a reduced concentration of oil. The miscella stream may be processed to separate the extracted oil from the solvent, such as by passing the stream through one or more distillation columns and/or stripping vessels. The solvent-wet solids may also be processed to dry the solids and remove residual solvent. These and other processes within the solvent extraction system may generate exhaust gases containing residual organic solvent. For example, steam used to vaporize solvent in a distillation column, stripping vessel, and/or solids desolventizer may be passed through a condenser (e.g., evaporator condenser, vent condenser), to recover condensable liquids. The residual non-condensable gas with any entrained organic solvent may form exhaust gas needing further processing.

In some configurations according to the present disclosure, an absorption system is provided to process a gas stream, such as exhaust gas from a solvent extraction system, to remove entrained organic solvent from the gas stream. The absorption system may process the vent gas by conveying the vent gas in a countercurrent direction with an absorbent liquid and further conveying the vent gas in a co-current direction with an absorbent liquid. For example, the vent gas may be conveyed in a countercurrent direction with a first lean absorbent liquid to absorb solvent out of the gas stream to generate a first conditioned gas stream. The resulting first conditioned gas stream can then be conveyed in a co-current direction with a second lean absorbent liquid to absorb solvent out of the first conditioned gas stream and generate a second conditioned gas stream. The absorption system can be implemented using a single absorption column containing both a countercurrent flow zone and a co-current flow zone or by using two different vessels, one of which provides a countercurrent flow direction and the other of which provides a co-current flow direction.

By configuring the absorption system with countercurrent absorbent flow followed by co-current absorbent flow, this absorption system can provide different stages of when the gas being processed is contacted with absorbent to absorb solvent from the gas. The relative flow rates of gas and absorbent in each of the two stages can be independently controlled. For example, in the first countercurrent flow stage, the ratio of absorbent to vent gas may be comparatively low. This can allow the gas to suitably contact and intermix with the absorbent without flooding the absorption column. In the second co-current flow stage, the ratio of absorbent to vent gas may be increased relative to the first countercurrent flow stage. For example, in the second stage, the flow rate of absorbent may be significantly increased relative to the first countercurrent stage while passing substantially the same flow rate of gas through both stages.

Configuring the absorption system with a first countercurrent flow stage can efficiently absorb a majority of the residual solvent from the vent gas entering the system, while providing a second co-current flow stage with increased absorbent flow rate can remove an additional amount of solvent from the vent gas before discharge. As a result, the additional second co-current flow stage can effectively “polish” the vent gas, further reducing the amount of residual solvent in the vent gas. This can better purify the vent gas for further processing and/or environmental discharge.

In one example, a method of absorbing a solvent from a gas stream is described. The method includes flowing a gas stream comprising air and a hydrocarbon through an absorption column in a countercurrent direction with a first lean absorbent liquid to absorb solvent out of the gas stream and generate a first conditioned gas stream and a first rich absorbent liquid. The method also includes flowing the first conditioned gas stream through the absorption column in a co-current direction with a second lean absorbent liquid to absorb solvent out of the first conditioned gas stream and generate a second conditioned gas stream having a reduced concentration of the hydrocarbon than the first conditioned gas stream and a second rich absorbent liquid. The example method can also include discharging the second conditioned gas stream from the absorption column.

In another example, an absorption system is described that includes a vertical column divided into a countercurrent flow zone and a co-current flow zone. The vertical column includes a gas stream inlet, a first absorbent inlet, a transition zone, a second absorbent inlet, and a gas outlet. According to the example, the gas stream inlet is configured to receive a gas stream comprising air and a hydrocarbon and supply the gas stream to the countercurrent flow zone. The first absorbent inlet is configured to receive a first lean absorbent liquid and supply the first lean absorbent liquid to the countercurrent flow zone in a countercurrent direction from the gas stream, the first lean absorbent liquid absorbing solvent out of the gas stream to generate a first conditioned gas stream and a first rich absorbent liquid. The transition zone connects the countercurrent flow zone and the co-current flow zone. According to the example, the transition zone is configured to receive the first conditioned gas stream exiting the countercurrent flow zone and supply the first conditioned gas stream to the co-current flow zone. The second absorbent inlet is configured to receive a second lean absorbent liquid and supply the second lean absorbent liquid to the co-current flow zone in a co-current direction with the first conditioned gas stream, the second lean absorbent liquid absorbing solvent out of the first conditioned gas stream to generate a second conditioned gas stream and a second rich absorbent liquid. The gas outlet is configured to discharge the second conditioned gas stream from the vertical column.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram illustrating an example extraction system according to the disclosure.

FIG. 2 is a block diagram illustrating an example absorption system that can be used in conjunction with the example extraction system of FIG. 1.

FIG. 3 is a sectional view of an example configuration of an absorption system in which the absorption system is implemented as a single column or vessel divided into different flow zones.

### DETAILED DESCRIPTION

In general, this disclosure relates to liquid-solid extractor systems and processes, including solvent recovery systems and processes. In some examples, an oleaginous material is processed in a continuous flow extractor that conveys a continuous flow of material from its inlet to its outlet while an organic solvent is conveyed in a countercurrent direction from a solvent inlet to a solvent outlet. As the solvent is conveyed from its inlet to its outlet, the concentration of extracted oil relative to solvent increases from a relatively small extract-to-solvent ratio to a comparatively large extract-to-solvent ratio. Similarly, as the solid material is conveyed in the opposing direction, the concentration of extract in the solid feedstock decreases from a comparatively high concentration at the inlet to a comparatively low concentration at the outlet.

The organic solvent discharged from the extractor, which may be referred to as a miscella, contains extracted oil from the oleaginous feedstock. The residual solid material discharged from the extractor contains solvent-wet solids having a reduced concentration of oil as compared to the original oleaginous feedstock. The miscella can be processed in a solvent recovery unit to vaporize the organic solvent from the extracted oil. The residual solid material can be processed in a desolventizer unit to vaporize the organic solvent from the solid material. Vapor stream(s) from the solvent recovery unit and/or desolventizer unit may be condensed to remove condensed water and/or organic solvent, producing an exhaust gas stream containing air and residual organic solvent.

In some examples of the present disclosure, the exhaust gas stream can be processed in an absorption system to help remove residual organic solvent from the gas stream, e.g., before discharging the gas stream to the atmosphere. The exhaust gas stream containing air and residual organic solvent can be passed through two stages of absorption: a countercurrent stage and a co-current stage. In the countercurrent stage, the gas stream can flow in an opposite direction a flow of absorbent contacting the gas stream. In the co-current stage, the gas stream can flow in the same direction as the flow of absorbent contacting the gas stream. Organic solvent in the exhaust gas stream can absorb into the absorbent in the countercurrent stage and in the co-current stage, e.g., producing a substantially solvent-free exhaust gas stream. The amount of organic solvent present in the exhaust gas stream may initially be reduced in the countercurrent stage, producing a first conditioned gas stream that has a reduced concentration of organic solvent as compared to the incoming gas stream supplied to the countercurrent stage. This first conditioned gas stream can then be supplied to the co-current stage to further reduce the amount of organic solvent present in the gas stream, producing a second conditioned gas stream that has a reduced concentration of organic solvent as compared to the incoming first conditioned gas stream.

Processing the gas stream using a combination of countercurrent flow and co-current flow can be beneficial to improve the absorption efficiency of the absorption system and reduce the amount of residual organic solvent in the process gas stream. Flow rates in the countercurrent stage in the co-current stage may be independently controlled, e.g., providing different absorbent flow rates and/or different gas stream flow rates in each stage to help maximize processing efficiency. For example, the absorbent flow rate in the countercurrent stage may be controlled to provide good liquid-gas mixing without flooding the absorption column in that stage. The absorbent flow rate in the co-current stage may be increased, e.g., to increase the amount of absorbent relative to the amount of gas for final scrubbing prior to discharging the gas stream to the atmosphere.

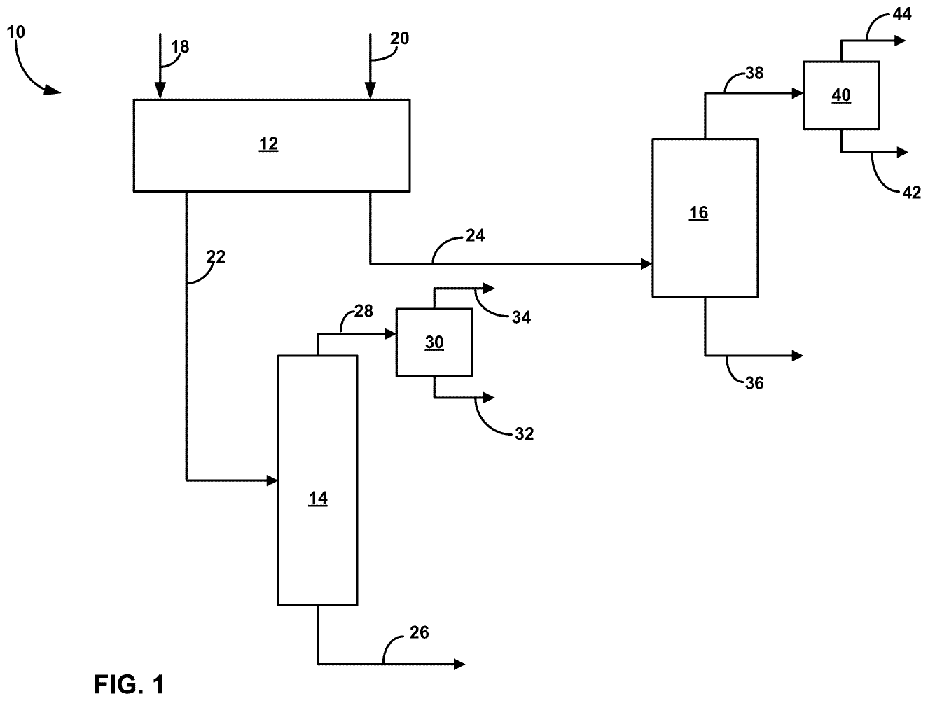

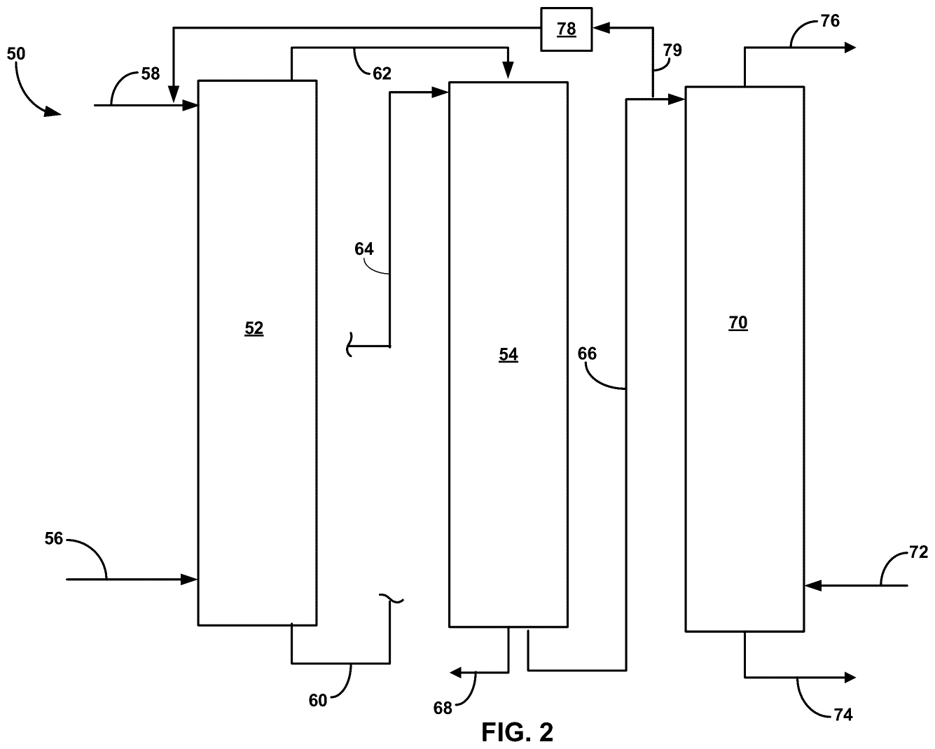

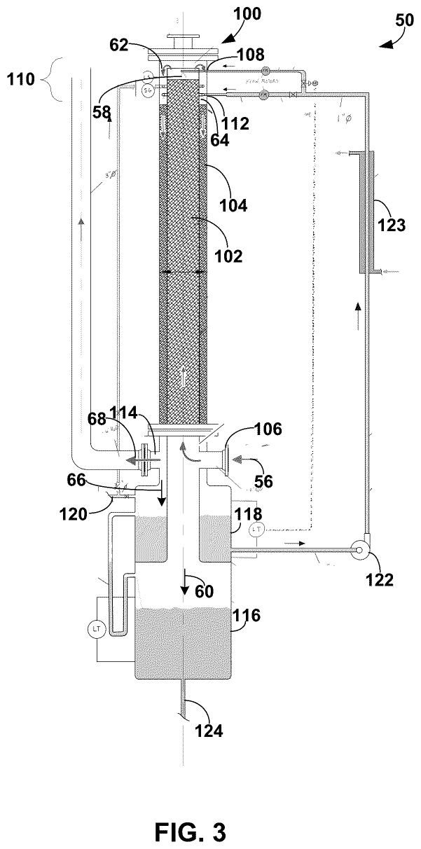

Example absorption systems and techniques will be described in greater detail with respect to FIGS. 2 and 3. However, an example extraction system that may generate vent gases containing organic solvent to be removed via an absorption system will first be described with respect to FIG. 1.

FIG. 1 is a block diagram illustrating an example extraction system 10 according to the disclosure. System 10 includes an extractor 12, a solvent recovery unit 14, and a desolventizer unit 16. In operation, a feedstock 18 containing a material to be extracted is introduced into a feed inlet of the extractor 12 and solvent 20 is introduced into a solvent inlet of the extractor. The feedstock 18 is contacted with the solvent 20 within the extractor 12 to extract oil from the feedstock into the solvent. The extractor can produce a miscella stream 22 containing extracted oil in organic solvent. The extractor can also produce a solvent-wet residual solids stream 24 containing residual solid material from the feedstock whose oil content has been extracted.

In the example configuration of system 10, the miscella stream 22 is conveyed to solvent recovery unit 14. The miscella stream 22 can be processed within the solvent recovery unit to separate extracted oil from the organic solvent. For example, solvent recovery unit 14 may receive steam that vaporizes solvent from the miscella stream 22. Solvent recovery unit 14 can produce an extracted oil stream 26 substantially or entirely devoid of organic solvent and a first recovered solvent stream 28. Organic solvent within the first recovered solvent stream 28 may be vaporized solvent mixed with air and/or steam (e.g. providing a gas phase stream). In some examples, the first recovered solvent stream 28 is passed through a condenser 30 to remove condensable liquids 32, including recovered solvent, yielding a residual stream of non-condensable exhaust gas 34. This first exhaust gas 34 may contain residual and/or entrained organic solvent that does not condense within the condenser.

Solvent-wet solids stream 24 in the example configuration of FIG. 1 is conveyed to the desolventizer unit 16. The desolventizer unit 16 can dry the solvent wet solids discharged from extractor 12. For example, desolventizer unit 16 may apply heat either directly or indirectly to the solvent wet solids, such as using forced air and/or steam, to vaporize solvent from the surface of the solids. The desolventizer unit 16 can produce a dried residual solids stream 36 and a second recovered solvent stream 38. Organic solvent within the second recovered solvent stream 38 may be vaporized solvent mixed with air and/or steam (e.g. providing a gas phase stream). The second recovered solvent stream 38 may be passed through a condenser 40 to remove condensable liquids 42, including recovered solvent from the solvent wet solids, yielding a residual stream of non-condensable exhaust gas 44. The second exhaust gas 44 may contain residual and/or entrained organic solvent that does not condense within the condenser.

Extractor 12 can be implemented using any suitable type of extractor configuration. In different examples, extractor 12 may be an immersion extractor, a percolation extractor, or yet other type of extractor design. Independent of the specific configuration of extractor 12, the extractor may be configured to operate so the feedstock stream 18 and solvent stream 20 flow in countercurrent directions through a housing of the extractor. For example, fresh oil-bearing feedstock 18 may flow through one inlet of the extractor housing while fresh solvent substantially or entirely devoid of extracted oil is passed through a second inlet of the extractor. As solvent travels through the extractor housing from the solvent inlet to the miscella outlet, the solvent can flow in a countercurrent direction to the flow of solid material passing from the feedstock inlet to the residual solids outlet. The solvent can intermix with the oil-bearing material within the extractor, causing oil and/or other extractable components to extract out of the solid feedstock into the solvent. The concentration of extract (e.g., oil) relative to solvent increases from a relatively small extract-to-solvent ratio to a comparatively large extract-to-solvent ratio. Similarly, as the solid feedstock is conveyed in the opposing direction, the concentration of extract in the solid feedstock decreases from a comparatively high concentration at the inlet to a comparatively low concentration at the outlet.

Extractor 12 can process any desired oleaginous feedstock using any suitable extraction fluid. Example types of oleaginous materials that can be processed using extractor 12 include, but are not limited to, soybeans (and/or soy protein concentrate), rapeseed, hemp, sunflower seed, peanuts, cottonseed, palm kernels, and corn germ and combinations thereof, as well as other oil-bearing seeds and fruits. Solvents that can be used to extract oil contained within the oleaginous material being processed are generally organic solvents, such as acetone, hexane, toluene, and/or alcohol-based solvents (e.g., ethanol). Typical oleaginous materials processed using extractor 12 are plant-based materials, yielding a triglyceride vegetable oil as an extracted oil product.

Solvent recovery unit 14 processing miscella stream 22 to separate extracted oil from solvent can be implemented using one or more separation units. For example, solvent recovery unit 14 may be implemented using one or more distillation columns, stripping columns, and/or evaporator units. Independent of the specific configuration of solvent recovery unit 14, the solvent recovery unit may be effective to remove substantially all of the organic solvent from the extracted oil in the miscella stream 22. For example, solvent recovery unit 14 may produce extracted oil stream 26 having less than 5 weight percent organic solvent, such as less than 3 weight percent organic solvent, less than 1 weight percent organic solvent, or less than 0.5 weight percent organic solvent.

Desolventizer unit 16 processing residual solids stream 24 can also be implemented using one or more separation units. For example, desolventizer unit 16 may be configured as a desolventizer toaster or other desolventizing device that increases the temperature the solids stream 24. The temperature of the stream may be increased to a temperature above the boiling point of the solvent introduced into extractor 12, causing residual solvent to vaporize. In some configurations, steam is injected into desolventizer unit 16 in addition to or in lieu of any other direct or indirect heating.

Exhaust gases produced by extraction system 10 can contain residual solvent not condensed at the operating conditions at which condenser 30, 40 operate. For example, extraction system 10 can produce first exhaust gas 34 from solvent recovery unit 14 containing residual organic solvent not condensed in condenser 30. Similarly, extraction system 10 can produce second exhaust gas 44 from desolventizing unit 16 contain residual organic solvent not condensed in condenser 40. Exhaust gases produced by extraction system 10 can be processed in an absorption system to remove residual solvent carried by the exhaust gases to prepare the exhaust gases for subsequent discharge. For example, first exhaust gas 34 and/or second exhaust gas 44 produced by extraction system 10 can be sent to one or more absorption units to remove organic solvent carried by the exhaust gas. In some implementations, the exhaust gases produced by extraction system 10, including first exhaust gas 34 and second exhaust gas 44, may be combined together to produce a composite exhaust gas stream that is fed to an inlet of an absorption system for combined processing. In either case, organic solvent carried by the exhaust gas can be absorbed by an absorbent liquid in the absorption system, e.g., reducing the concentration of residual organic solvent in the exhaust gas to a level suitable (e.g., according to local environmental regulations) to allow the exhaust gas to be discharged to atmosphere.

FIG. 2 is a block diagram illustrating an example absorption system 50 that can be used in conjunction with extraction system 10 from FIG. 1. In the illustrated example, absorption system 50 includes a first absorption column 52, a second absorption column 54, and a stripping column 70. First absorption column 52 may operate as a countercurrent stage in which an absorbent liquid flows in a countercurrent direction to a gas stream being processed. Second absorption column 54 may operate as a co-current stage in which an absorbent liquid flows in a co-current direction with the gas stream being processed. Stripping column 70 may strip absorbed organic solvent from the absorbent liquid used in first absorption column 52 and/or second absorption column 54 to regenerate the absorbent liquid for reuse in absorption system 50. Although the example configuration shows two absorption columns and a single stripping column, it should be appreciated that practical implementations of such a system may include one or more absorption columns 52 and/or one or more stripping columns 70. For example, as will be described with respect to FIG. 3, absorption system 50 may be implemented using a single absorption column that includes a countercurrent stage and a co-current stage in lieu of utilizing to different absorption columns as shown in FIG. 2.

In the example of FIG. 2, first absorption column 52 can receive exhaust gases 56 containing residual organic solvent from one or more processing units of extraction system 10. For example, first absorption column 52 may receive first exhaust gas 34 from solvent recovery unit 14, second exhaust gas 44 from desolventizer unit 16, and/or gases from any other processing unit within the extraction system generating exhaust gas. Exhaust gas streams from different processing units may be combined together and processed as a single stream by first absorption column 52, or separate exhaust gas streams from separate processing units may be processed in different absorption columns. Typically, exhaust gas delivered to first absorption column 52 will have first been passed through a condenser 30, 40 to remove condensable gases and/or liquids from the gas stream. As a result, the exhaust gas being processed by the absorption column may be cooled, non-condensable gas.

To remove residual organic solvent from the exhaust gas stream 56, the exhaust gas stream may be introduced into first absorption column 52 along with a first lean absorbent liquid 58. For example, exhaust gas 56 may be introduced into a substantially opposite end of the vertical first absorption column 52 from an end of the column in which the first lean absorbent liquid 58 is introduced in the column. The exhaust gas 56 and first lean absorbent liquid 58 can flow in countercurrent directions along the length of first absorption column 52. For example, first lean absorbent liquid 58 may be pressurized through one or more pumps and delivered to an elevated location of first absorption column 52, allowing the first lean absorbent liquid to flow downwardly with respect to gravity. Exhaust gas 56 may be pressurized and may flow upwardly with respect to gravity in a countercurrent direction relative to the flow direction of the first lean absorbent liquid 58.

Within first absorption column 52, organic solvent contained in exhaust gas 56 can transfer from the gas phase to the liquid phase. Organic solvent in exhaust gas 56 can be absorbed by first lean absorbent liquid 58, increasing the concentration of the organic solvent in the first lean absorbent liquid as the first lean absorbent liquid travels from an inlet to an outlet of the absorption column. The resulting absorbent liquid with increased concentration of organic solvent can be referred to as a first rich absorbent liquid 60 and can discharge from first absorption column 52 through a first rich absorbent liquid outlet. As exhaust gas 56 passed through first absorption column 52, the concentration of organic solvent in the gas is reduced as the solvent is absorbed by the liquid absorbent. The resulting exhaust gas stream with reduced concentration of organic solvent can be referred to as a first conditioned gas stream 62 and can discharge from first absorption column 52 through a first conditioned gas stream outlet.

In the example of FIG. 2, absorption system 50 includes a second absorption column 54 located downstream from first absorption column 52 and connected to the first absorption column via piping. Second absorption column 54 can receive the exhaust gas stream after the exhaust gas stream has been processed in first absorption column 52 and can contact the exhaust gas stream with a second lean absorbent liquid 64 to absorb additional residual solvent out of the gas stream. Second absorption column 54 may be implemented to convey the exhaust gas stream discharging from first absorption column 52 along with a second lean absorbent liquid in a co-current direction through the column.

For example, to remove residual organic solvent, first conditioned gas stream 62 discharging from first absorption column 52 may be introduced into second absorption column 54. When second absorption column 54 is implemented so that first conditioned gas stream 62 and second lean absorbent liquid 64 flow in a co-current direction, the two streams may be introduced at a same end of the vertical second absorption column 54. For example, first conditioned gas stream 62 and second lean absorbent liquid 64 may be introduced at an upper end of second absorption column 54 under pressure and cause to flow downwardly in a co-current direction along the length of the column. For example, second lean absorbent liquid 64 may be pressurized through one or more pumps and delivered to an elevated location of second absorption column 54, allowing the second lean absorbent liquid to flow downwardly with respect to gravity. First conditioned gas stream 62 may also be pressurized and be introduced to an elevated location of second absorption column 54 and caused to flow downwardly with respect to gravity in a co-current direction relative to the flow direction of the second lean absorbent liquid 64.

Within second absorption column 54, residual organic solvent contained in first conditioned gas stream 62 can transfer from the gas phase to the liquid phase. Organic solvent in first conditioned gas stream 62 can be absorbed by second lean absorbent liquid 64, increasing the concentration of the organic solvent in the second lean absorbent liquid as the second lean absorbent liquid travels from an inlet to an outlet of the absorption column. The resulting absorbent liquid with increased concentration of organic solvent can be referred to as a second rich absorbent liquid 66 and can discharge from second absorption column 54 through a second rich absorbent liquid outlet. As first conditioned gas stream 62 passes through second absorption column 54, the concentration of organic solvent in the gas is reduced as the solvent is absorbed by the liquid absorbent. The resulting gas stream with reduced concentration of organic solvent can be referred to as a second conditioned gas stream 68 and can discharge from second absorption column 54 through a second conditioned gas stream outlet. One or more disentrainment devices such as baffles, a cyclone, and/or a demister pad can be used to reduce potential liquid carryover with the gas stream. In either case, the second condition gas stream 68 may be discharged to the atmosphere and/or otherwise processed.

Absorption of organic solvent from the gas phase to the liquid phase in first absorption column 52 and second absorption column 54 may be effective to remove a sufficient amount of organic solvent to allow second condition gas stream 68 to be further processed and/or discharged to atmosphere. In some examples, exhaust gas 56 entering absorption column 52 may have from 30 weight percent to 70 weight percent organic solvent. By contrast, after being contacted with a first absorbent liquid in first absorption column 52 and a second absorbent liquid and second absorption column 54, second condition gas stream 68 may have less than 15 weight percent organic solvent, such as less than 10 weight percent organic solvent, less than five weight percent organic solvent, less than three weight percent organic solvent, less than one weight percent organic solvent, less than 0.5 weight percent organic solvent, less than 0.25 weight percent organic solvent, or less than 0.1 weight percent organic solvent.

In practice, a majority of the weight of the solvent remove from the gas stream being processed by absorption system 50 may be removed in first absorption column 52 with a comparatively lesser amount of solvent removed by second absorption column 54. For example, a ratio of the amount of solvent removed from the gas stream by first absorption column 52 (e.g., the amount of solvent in exhaust gas 56 minus the amount of solvent in first conditioned gas stream 62) divided by the amount of solvent removed from the gas stream by second absorption column 54 (e.g., the amount of solvent in first conditioned gas stream 62 minus the amount of solvent in second condition gas stream 68) may be greater than 1.0, such as greater than 2.0, greater than 3.0, greater than 4.0, or greater than 5.0.

The amount of solvent removed from the gas stream by first absorption column 52 and second absorption column 54 may be controlled, e.g., based on the sizing and configuration of the two columns and/or the flow rates through the two columns. The gas flow rate and/or absorbent liquid flow rate through first absorption column 52 and second absorption column 54 may be independently controllable to adjust the relative flow rates of gas and absorbent liquids to the two different columns. For instance, in some implementations, the ratio of absorbent flow rate to gas flow rate in first absorption column 52 may be comparatively low when flowing in the countercurrent direction but may be increased in second absorption column 54 when flowing in the co-current direction. In some examples, second absorption column 54 may operate using absorbent flow rates that would cause flooding in first absorption column 52, if the first absorption column was operated at those flow rates, but which are permissible because of the co-current flow direction in the second absorption column.

Increasing the ratio of absorbent flow rate to gas flow rate in the second absorption column 54 compared to first absorption column 52 can increase the amount of organic solvent absorbed from the gas stream (e.g., preparing the gas stream for final discharge). In some examples, such as where the flow rate of gas through first absorption column 52 and second absorption column 54 is substantially the same, the flow rate of the second lean absorbent liquid 64 may be greater than the flow rate of the first lean absorbent liquid 58.

For example, a ratio of a flow rate of the first lean absorbent liquid divided by a flow rate of the second lean absorbent liquid ranges may be less than 1.0, such as less than 0.8, or from 0.3 to 0.6. When the gas flow through the first absorption column 52 is substantially the same as the gas flow through the second absorption column 54 (the flow rate of gas stream 56 is substantially the same as the flow rate of first conditioned gas stream 62), the flow rate of the second lean absorbent may be at least 25% larger than the flow rate of the first lean absorbent liquid, such as at least 50% larger, at least 75% larger, at least 100% larger, or at least 150% larger. For example, the flow rate of the second lean absorbent liquid may within a range from 1.25 to 3.5 times the flow rate of the first lean absorbent liquid, such as within a range from 1.5 to 2.5 times the flow rate of the first lean absorbent liquid, or from 1.5 to 2.0 times the flow rate of the first lean absorbent liquid.

A variety of different liquids may be used as an absorbent liquid for first absorption column 52 and second absorption column 54. In one example, mineral oil is used as the absorbent liquid for both absorption columns. In another example, extracted oil produced by extractor 12 (FIG. 1) and recovered as extracted oil stream 26 may be used as the absorbent liquid for both absorption columns. Other example absorbent liquids may include aqueous absorbent liquids, such as water (e.g., the absorbent liquid may consist of or consist essentially of water) when the solvent used in the extraction process is an alcohol-based solvent (e.g., anhydrous ethanol; ethanol and water mixture). In typical practice, first absorption column 52 and second absorption column 54 may use the same type of absorbent liquid (e.g., mineral oil), although different absorbent liquids having different chemical compositions can be used for each absorption column in other applications.

In some implementations, the absorbent liquid (e.g., first lean absorbent liquid 58 and/or second lean absorbent liquid 64) is temperature adjusted prior to contacting a corresponding gas stream. For example, the absorbent liquid may be heated to a temperature greater than the temperature of a gas stream that the absorbent liquid is to contact, such as a temperature at least 5 degrees hotter than the temperature of the gas stream, at least 10 degrees hotter, at least 20 degrees hotter, at least 30 degrees hotter, or at least 50 degrees hotter. The first lean absorbent liquid 58 may be heated to the same temperature as the second lean absorbent liquid 64 or may be heated to a different temperature (e.g., a hotter temperature) than the second lean absorbent liquid 64.

First absorption column 52 and second absorption column 54 can each have a variety of different configurations. In some examples, one or both absorption columns include one or more packed beds along with corresponding support plates and liquid distribution hardware to cause the exhaust gas and extracted oil absorption liquid to intermix. Additionally or alternatively, one or both absorption columns may be configured as a trayed column which may or may not have one or more packed beds sections as well. In a trayed column configuration, the absorption column can include a plurality of trays vertically spaced from one another. Each tray may have openings that allow the passage of gas from one tray to an adjacent tray and a downcomer that directs the flow of liquid from one tray to an adjacent tray. For example, each tray may be configured as a valve tray with movable or fixed valves. A fixed valve tray configuration may be useful to provide good contact between the rising exhaust gas and falling oil used as the absorption medium while helping to minimize performance degradation due to fouling and other service life issues.

After the gas stream being processed has passed through second absorption column 54, the resulting second condition gas stream 68 may be discharged to atmosphere or directed to other suitable downstream processing. The absorbent liquid containing organic solvent absorbed from the gas (e.g., first rich absorbent liquid 60 and/or second rich absorbent liquid 66) can be processed in stripping column 70 to remove the absorbed organic solvent. In some configurations, stripping gas such as steam 72 is introduced into the stripping column 70 to strip the organic solvent from the oil absorption medium. Accordingly, stripping column 70 may produce a recovered absorbent liquid 74 substantially or entirely devoid of organic solvent absorbed from the exhaust gas stream. Stripping column 70 may also produce a recovered solvent stream 76 containing solvent vapor intermixed with stripping gas that can then be sent to a condenser for condensing and further processing. Although not illustrated, an oil heater and/or other processing equipment may process the rich absorbent liquid before entering stripping column 70.

In some implementations, the first rich absorbent liquid 60 and the second rich absorbent liquid 66 are each sent to one or more stripping columns 70 to be stripped of organic solvent to regenerate the lean first absorbent liquid 58 and the lean second absorbent liquid 64, respectively. In some examples, at least a portion of the second rich absorbent liquid 66 is recycled to function as the first lean absorbent liquid 58. Because the concentration of solvent absorbed into the second rich absorbent liquid 66 is typically less than the concentration of solvent absorbed into the first lean absorbent liquid 60, the second rich absorbent liquid 66 can be recycled to function as the first lean absorbent liquid 58 before stripping in stripping column 70.

For instance, in the example, configuration of FIG. 2, the second rich absorbent liquid 66 generated by second absorption column 54 is recycled to function as first lean absorbent liquid 58 for first absorption column 52. Absorption system 50 may include an absorbent reservoir 78 that second rich absorbent liquid 66 is discharged into and from which the first lean absorbent stream is drawn and supplied to first absorption column 52. When the second absorption column 54 is operated with a high absorbent flow rate than the first absorption column 52, an amount of the second rich absorbent liquid 66 in excess of what is needed to fulfill the supply requirements for the first lean absorbent 58 may be separately processed. For example, the excess amount of the second rich absorbent liquid 66 may be delivered to a second absorbent reservoir (e.g., via line 79) and/or stripping column 70 to be stripped.

As briefly discussed above, absorption system 50 that includes a countercurrent absorption stage and a co-current absorption stage in which a gas stream being processed is flowed countercurrent and co-current, respectively, with one or more absorbent liquids can be implemented in a variety of different ways. FIG. 2 illustrates absorption system 50 implemented with two different absorption columns that are physically separate vessels fluidly connected to each other. In other examples, a single absorption column divided into different zones, including a countercurrent zone and a co-current zone, may be used to implement the processing principles discussed herein. Configuring absorption system 50 is a single absorption column divided internally into different flow zones may be beneficial to reduce the footprint and amount of space needed to deploy the absorption system.

FIG. 3 is a sectional view of an example configuration of absorption system 50 in which the absorption system is implemented as a single column or vessel divided into different flow zones. In particular, in the illustrated example, absorption system 50 is illustrated as being implemented as a single column 100 divided into a countercurrent flow zone 102 and a co-current flow zone 104. Column 100 may be oriented vertically with respect to gravity, e.g., such that the gas being processed flows upward against a downward flow of absorbent liquid in the countercurrent flow zone and the gas being processed and the absorbent liquid both flow downward in the countercurrent flow zone. Countercurrent flow zone 102 and co-current flow zone 104 of absorption system 50 in FIG. 3 can utilize the feeds (e.g., gas and absorbent liquid compositions and flow rates), processing characteristics (e.g., absorbent flow rate, gas flow rate), absorption column internals, and exhibit the performance characteristics (e.g., solvent removal characteristics) discussed above with respect to first absorption column 52 and second absorption column 54, respectively.

In the example of FIG. 3, column 100 is illustrated as having a gas stream inlet 106 configured to receive gas stream 56 from extractor system 10 (FIG. 1). Gas stream 56 can include air and hydrocarbon (e.g. organic solvent), as discussed above. Gas stream inlet 106 can supply gas stream to countercurrent flow zone 102 of the column for countercurrent flow and contact with a first lean absorbent liquid.

To supply the first lean absorbent liquid to countercurrent flow zone 102, column 100 can include a first absorbent inlet 108. The first absorbent inlet 108 can receive first lean absorbent liquid 58 and supply the first lean absorbent liquid to countercurrent flow zone 102 in a countercurrent direction from gas stream 56. Within countercurrent flow zone 102, the first lean absorbent liquid 58 can absorb solvent out of the gas stream to generate a first conditioned gas stream 62 and a first rich absorbent liquid 60.

To connect the countercurrent flow zone 102 with the co-current flow zone 104, column 100 can include a transition zone 110 connecting the two flow zones. Transition zone 110 can receive the first conditioned gas stream 62 exiting countercurrent flow zone 102 and supply the first conditioned gas stream to co-current flow zone 104. Transition zone 110 may be a space within column 100 where countercurrent flow zone 102 and co-current flow zone 104 fluidly connect and fluid (e.g., gas) from the countercurrent flow zone transitions into the co-current flow zone.

With further reference to the example configuration of FIG. 3, column 100 can include a second absorbent inlet 112 configured to receive a second lean absorbent liquid 64. Second absorbent inlet 112 can supply the second lean absorbent liquid 64 to the co-current flow zone 104 in a co-current direction with first conditioned gas stream 62. In some implementations, the second lean absorbent liquid 64 that is used to absorb additional organic solvent from the first conditioned gas stream 62 comprises some or all of the first rich absorbent liquid 60 carried over from the countercurrent flow zone 102 supplemented with an additional amount of absorbent liquid. This can provide an increased amount (e.g., rate) of the second lean absorbent liquid 64 relative to the amount (e.g., rate) of the first lean absorbent liquid 58 supplied to the countercurrent flow zone 102. Within the co-current flow zone 104, the second lean absorbent liquid 64 can absorb solvent out of the first conditioned gas stream 62 to generate a second conditioned gas stream 68 and a second rich absorbent liquid 66. Column 100 can include a gas outlet 114 to discharge the second conditioned gas stream 68 from the column.

As noted, column 100 may be oriented vertically with respect to gravity, e.g., such that the gas being processed flows upward against a downward flow of absorbent liquid in the countercurrent flow zone and the gas being processed and the absorbent liquid both flow downward in the countercurrent flow zone. For example, column 100 can have a gas stream inlet adjacent a bottom of the vertical column, first absorbent inlet 108 can be positioned adjacent a top of the vertical column, and second absorbent inlet 112 can be positioned adjacent the top of the vertical column.

To supply absorption liquids to the countercurrent flow zone 102 and the co-current flow zone 104 of column 100, absorption system 50 may include a first absorption liquid reservoir 116 and a second absorption liquid reservoir 118. The first absorption liquid reservoir 116 can be fluidly connected to countercurrent flow zone 102 and positioned to receive the first rich absorbent liquid 60 discharged from the countercurrent flow zone. The second absorption liquid reservoir 118 can be fluidly connected to the co-current flow zone 104 and configured to receive the second rich absorbent liquid 66 discharged from the co-current flow zone. First absorption liquid reservoir 116 and second absorption liquid reservoir 118 or both illustrated as being positioned vertically below the countercurrent flow zone 102 and a co-current flow zone 104 of column 100. In particular, second absorption liquid reservoir 118 is illustrated as being vertically stacked relative to first absorption liquid reservoir 116 (with the second absorption liquid reservoir being positioned at a vertically elevated location above the first absorption liquid reservoir). Other reservoir arrangements can be implemented without departing from the scope of the disclosure.

In some examples, the second absorption liquid reservoir 118 includes a fresh absorbent inlet 120 that receives a supply of fresh absorbent liquid (e.g., from stripping column 70) to the second absorption liquid reservoir. This can provide a mixture of the fresh absorbent liquid and the second rich absorbent liquid 66. A pump 122 can draw the mixture of the fresh absorbent liquid and the second rich absorbent liquid from the second absorption liquid reservoir 118 and supply the mixture as the first lean absorbent liquid 58 to the first absorbent inlet 108 and as the second lean absorbent liquid 64 to the second absorbent inlet 112. A heater 123 (e.g., jacketed line) can heat the absorbent liquid to a target temperature. First absorption liquid reservoir 116 can have an outlet 124 in fluid communication with stripping column 70.

In the illustrated arrangement, the countercurrent flow zone 102 is illustrated extending parallel to the co-current flow zone 104 along a length of the vertical column. For example, column 100 may define an annulus having an inner cylinder and an outer ring. The countercurrent flow zone 102 can be configured as the inner cylinder and the co-current flow zone 104 the outer ring (or vice versa). In alternative implementations, column 100 may be partitioned along its length (e.g., without defining an annular space) and the countercurrent flow zone 102 and co-current flow zone 104 may be defined by different partitioned sections of the vessel.

Various examples have been described. These and other examples are within the scope of the following claims.

1. A method of absorbing a solvent from a gas stream, the method comprising:
flowing a gas stream comprising air and a hydrocarbon through an absorption column in a countercurrent direction with a first lean absorbent liquid to absorb solvent out of the gas stream and generate a first conditioned gas stream and a first rich absorbent liquid;
flowing the first conditioned gas stream through the absorption column in a co-current direction with a second lean absorbent liquid to absorb solvent out of the first conditioned gas stream and generate a second conditioned gas stream having a reduced concentration of the hydrocarbon than the first conditioned gas stream and a second rich absorbent liquid; and
discharging the second conditioned gas stream from the absorption column.

2. The method of claim 1, wherein the first lean absorbent liquid and the second lean absorbent liquid are a same absorbent.

3. The method of claim 1, wherein the absorption column is fluidly connected to a first absorption liquid reservoir and a second absorption liquid reservoir, and further comprising:
discharging the first rich absorbent liquid into the first reservoir; and
discharging the second rich absorbent liquid into the second reservoir.

4. The method of claim 3, further comprising introducing fresh absorbent liquid into the second reservoir to provide a mixture of the fresh absorbent liquid and the second rich absorbent liquid, wherein:
flowing the gas stream through the absorption column in the countercurrent direction with the first lean absorbent liquid comprises drawing the mixture of the fresh absorbent liquid and the second rich absorbent liquid from the second reservoir and providing the mixture as the first lean absorbent; and
flowing the first conditioned gas stream through the absorption column in the co-current direction with the second lean absorbent liquid comprises drawing the mixture of the fresh absorbent liquid and the second rich absorbent liquid from the second reservoir and providing the mixture as the second lean absorbent.

5. The method of claim 1, further comprising stripping the first rich absorbent liquid to remove the hydrocarbon.

6. The method of claim 1, wherein the absorption column comprises a first absorption column through which the first lean absorbent liquid and the gas stream are conveyed in a countercurrent direction and a second absorption column through which the second lean absorbent liquid and the first conditioned gas stream are conveyed in a co-current direction.

7. The method of claim 1, wherein the absorption column comprises a single column divided into a countercurrent flow zone and a co-current flow zone.

8. The method of claim 1, wherein a ratio of a flow rate of the first lean absorbent liquid divided by a flow rate of the second lean absorbent liquid is less than 1.0.

9. The method of claim 1, wherein:
the absorption column comprises a vertically oriented column divided into a countercurrent flow zone and a co-current flow zone;
flowing the gas stream through the absorption column in the countercurrent direction with the first lean absorbent liquid comprises flowing the gas stream upwardly through the countercurrent flow zone and the first lean absorbent liquid downwardly through the countercurrent flow zone; and
flowing the first conditioned gas stream through the absorption column in the co-current direction with the second lean absorbent liquid comprises flowing the first conditioned gas stream and the second lean absorbent liquid downwardly through the co-current flow zone.

10. The method of claim 9, wherein the countercurrent flow zone extends parallel to the co-current flow zone along a length of the vertically oriented column.

11. The method of claim 9, wherein the vertically oriented column comprises an annulus having an inner cylinder and an outer ring, the countercurrent flow zone is the inner cylinder, and the co-current flow zone is the outer ring.

12. The method of claim 9, wherein the vertically oriented column further comprises a head space joining the countercurrent flow zone and the co-current flow zone, the first conditioned gas stream exiting the countercurrent flow zone and entering the co-current flow zone in the head space.

13. The method of claim 1, wherein flowing the gas stream through the absorption column and flowing the first conditioned gas stream through the absorption column comprises applying a vacuum to a discharge port of the absorption column drawing the second conditioned gas stream out of the absorption column.

14. The method of claim 1, wherein the hydrocarbon comprises hexane.

15. The method of claim 1, further comprising:
extracting a material to be processed with a solvent comprising the hydrocarbon in an extractor to generate a solvent-wet extracted material stream and a miscella stream;
desolventizing the solvent-wet extracted material stream in a desolventizer to generate a vapor stream and a desolventized extracted material stream; and
passing the vapor stream through a condenser to generate a condensed liquid and a non-condensed gas stream, the non-condensed gas stream forming at least a portion of the gas stream flowed through the absorption column.

16. An absorption system comprising:
a vertical column divided into a countercurrent flow zone and a co-current flow zone, the vertical column comprising:
a gas stream inlet configured to receive a gas stream comprising air and a hydrocarbon and supply the gas stream to the countercurrent flow zone;
a first absorbent inlet configured to receive a first lean absorbent liquid and supply the first lean absorbent liquid to the countercurrent flow zone in a countercurrent direction from the gas stream, the first lean absorbent liquid absorbing solvent out of the gas stream to generate a first conditioned gas stream and a first rich absorbent liquid;
a transition zone connecting the countercurrent flow zone and the co-current flow zone, the transition zone being configured to receive the first conditioned gas stream exiting the countercurrent flow zone and supply the first conditioned gas stream to the co-current flow zone;
a second absorbent inlet configured to receive a second lean absorbent liquid and supply the second lean absorbent liquid to the co-current flow zone in a co-current direction with the first conditioned gas stream, the second lean absorbent liquid absorbing solvent out of the first conditioned gas stream to generate a second conditioned gas stream and a second rich absorbent liquid; and
a gas outlet configured to discharge the second conditioned gas stream from the vertical column.

17. The system of claim 16, further comprising a first absorption liquid reservoir and a second absorption liquid reservoir, wherein the countercurrent flow zone is fluidly connected to the first absorption liquid reservoir and configured to discharge the first rich absorbent liquid into the first absorption liquid reservoir, and the co-current flow zone is fluidly connected to the second absorption liquid reservoir and configured to discharge the second rich absorbent liquid into the second absorption liquid reservoir.

18. The system of claim 17, wherein the second absorption liquid reservoir comprises a fresh absorbent inlet configured to supply fresh absorbent liquid to the second absorption liquid reservoir to provide a mixture of the fresh absorbent liquid and the second rich absorbent liquid and further comprising a pump configured to draw the mixture of the fresh absorbent liquid and the second rich absorbent liquid from the second absorption liquid reservoir and supply the mixture as the first lean absorbent liquid to the first absorbent inlet and as the second lean absorbent liquid to the second absorbent inlet.

19. An absorption system:
a first absorption column comprising a gas stream inlet configured to receive a gas stream comprising air and a hydrocarbon and a first absorbent inlet configured to receive a first lean absorbent liquid, wherein the gas stream inlet is positioned relative to the first absorbent inlet to cause the gas stream to zone in a countercurrent direction from the first lean absorbent liquid, and the first lean absorbent liquid is configured to absorb solvent out of the gas stream to generate a first conditioned gas stream and a first rich absorbent liquid; and
a second absorption column comprising a first conditioned gas stream inlet configured to receive the first conditioned gas stream from the first absorption column and a second absorbent inlet configured to receive a second lean absorbent liquid, wherein the first conditioned gas stream inlet is positioned relative to the second absorbent inlet to cause the first conditioned gas stream to flow in a co-current direction with the second lean absorbent liquid, and the second lean absorbent liquid is configured to absorb solvent out of the first conditioned gas stream to generate a second conditioned gas stream and a second rich absorbent liquid, wherein the second absorption column further comprises a gas outlet configured to discharge the second conditioned gas stream.

20. The absorption system of claim 19, further comprising:
a stripping column configured to receive one or both of the first rich absorbent liquid and the second rich absorbent liquid;
an extractor configured to extract a material to be processed with a solvent comprising the hydrocarbon to generate a solvent-wet extracted material stream and a miscella stream;
a desolventizer configured to receive the solvent-wet extracted material stream from the extractor and desolventize the solvent-wet extracted material stream to generate a vapor stream and a desolventized extracted material stream; and
a condenser configured to receive the vapor stream from the desolventize and condense the vapor stream to generate a condensed liquid and a non-condensed gas stream, the non-condensed gas stream being supplied to the gas stream inlet of the first absorption column.
